(19) United States
(12) Patent Application Publication
(10) Pub. No.: US 2020/0282117 A1
(43) Pub. Date: Sep. 10, 2020

(54) BREASTPUMP WITH FUNCTIONAL VACUUM PULSES

(71) Applicant: MEDELA HOLDING AG, Baar (CH)

(72) Inventors: Natalie Stevens, McHenry, IL (US); David Y. Cho, Arlington Heights, IL (US)

(21) Appl. No.: 16/807,723

(22) Filed: Mar. 3, 2020

Related U.S. Application Data
(60) Provisional application No. 62/815,135, filed on Mar. 7, 2019.

(51) Int. Cl. A61M 1/06; A61M 1/00

(52) U.S. Cl. CPC A61M 1/062; A61M 2210/1007; A61M 1/0068

(57) ABSTRACT

A method and system for enhancing expression of milk from breast alveoli includes providing a pump that delivers a pulsed vacuum. The pulsed vacuum may be delivered by a vacuum pump employing a rigid piston that operates as a multi-stroke accumulator pump. In an embodiment, a piston head of the rigid piston undergoes a non-linear motion relative to a sidewall of a cylinder of the pump, resulting in an oscillatory impulse on the interior of the vacuum chamber in the cylinder. The vacuum pulse frequency of the pump is applied over a cyclical vacuum frequency that would otherwise be applied had the piston head only moved up and down while maintaining a perpendicular relationship to the sidewall of the cylinder.

### CROSS-REFERENCE TO RELATED APPLICATIONS

This is the non-provisional, and claims the benefit of the filing date under 35 USC § 119(e), of U.S. Provisional Appl. No. 62/815,135, filed Mar. 7, 2019, which is hereby incorporated by reference in its entirety.

### BACKGROUND

### Field of the Disclosure

This disclosure relates generally to expression of milk and, more specifically, to a breastpump that generates and delivers a pulsed vacuum.

### Description of the Prior Art

Breast alveoli share many functional similarities with lung alveoli. In the case of lung alveoli, oxygen and carbon dioxide are transferred between the lungs and the bloodstream, with carbon dioxide being transferred to the air and then expulsed. In the case of breast alveoli, components of breastmilk, such as nutrients, are transferred from the bloodstream to milk and then expulsed. A known treatment of occurrences of respiratory distress syndrome in full-term and near-term infants is the application of high-frequency oscillatory ventilation (HFOV). The efficacy of this form of ventilation is attributable to the fact that HFOV improves oxygenation through the lung alveoli. HFOV operates on a principle of vacuum pulse generation.

Conventional electric breastpumps deliver a cyclical vacuum, regardless of whether the breastpump is a single-stroke or accumulator style of breastpump. While strides have been made to provide breast pumps that apply forces at frequencies to the nipples that mimic those delivered by a feeding infants, the volume of milk expressed during pumping sessions utilizing conventional breastpumps can be lower than the volume of milk delivered by direct feeding. Moreover, while the fat content of milk provides some of the greatest nourishing benefits of breastfeeding, the concentration of fat within breast milk has been found to increase toward the end of a feeding or pumping session. High fat concentration milk can be the most desirable, because higher fat concentration in milk leads to a more complete removal of the milk to the feeding infant, or a more effective pump. The physiology of epithelial milk-secreting cells, however, is such that they tend to close-off as the fat concentration of the milk increases, thereby preventing the delivery of high fat concentration milk to the feeding infant or, in the case of a breastpump, to a milk collection container.

### SUMMARY OF THE DISCLOSURE

Given the functional similarities of breast alveoli and lung alveoli, we have designed a breast pump that utilizes vacuum pulse generation. By providing vacuum pulse generation in addition to (or in lieu of) a conventional cyclical vacuum, breastpumps of the present disclosure provide improved milk removal from the breast alveoli and foster the maintenance of open apertures of epithelial milk-secreting cells to provide improved milk removal from breast alveioli.

Advantageously, breastpumps of the present disclosure deliver higher volumes of collected breast milk in a single pumping session, while applying a lower effective vacuum pressure to the breast.

In a preferred embodiment, a breastpump employs a multi-stroke accumulator with inherent vacuum steps. While conventional accumulator pumps employ elastomeric diaphragms, such pumps experience losses due to the dampening effects the diaphragm and its related support structure have on the vacuum pulses generated by the motor of such pumps. Instead of an elastomeric diaphragm, the instant breastpump uses a solid piston head sealed in a cylinder. During use, the stiff piston creates a distinct vacuum step, or pulse, that can be transferred through vacuum delivery tubing to a breast shield or related device, across a media separation membrane (if necessary), and ultimately to the breast pumping mother.

Though constructed as a linear piston/cylinder-style breastpump, the system of the present disclosure takes advantage of the nature of a multi-stroke pump style in which a reciprocating member generates small increments of vacuum that are accumulated together to reach a target vacuum.

In order to deliver vacuum pulses with a solid piston, it is found beneficial to actuate the solid piston in an eccentric manner. Due to the need to maintain the integrity of a pneumatic seal between the circumference of the piston head and the walls of the vacuum cylinder despite the eccentric movement of the solid piston, a low friction compression seal having a non-circular cross-section configuration, such as the MULTISEAL® bi-directional lip style seal available from Precision Associates, Inc. of Minneapolis, Minn., USA, is employed at the interface between the piston head and the sidewall of the vacuum cylinder.

The breastpump of the present disclosure applies a distinct vacuum pulse to stimulate the breast alveoli. The instant breastpump can achieve a higher vacuum than conventional diaphragm-style accumulator breastpumps operated under similar power conditions because energy losses attributable to material stretching are eliminated. Because a rigid piston can evacuate more volume in a chamber for each individual stroke, the breastpump of the present disclosure also can be constructed to have a smaller overall footprint than a diaphragm-style accumulator breastpump. Because diaphragm-style breastpumps are prone to fatigue, and even tears in the diaphragm membrane, the rigid piston-style pump of the present disclosure possesses superior reliability. Also, based on the strong correlation between the motor's current draw and the vacuum generated, vacuum control can be performed economically, without the need for costly pressure monitors. The mechanical architecture of a breastpump constructed in accordance with the teachings of the present disclosure is also tunable, in that the vacuum pulse frequency can be optimized by modifying the volume of the displacement chamber. The larger the chamber volume (which can be created either by using a larger cylinder cross-section or by increasing the piston stroke), the greater the vacuum step, and the smaller the chamber volume, the lower the vacuum step.

### DESCRIPTION OF THE SEVERAL VIEWS OF THE DRAWING

FIG. 1 is a semi-schematic view of a conventional diaphragm-style accumulator pump actuated by an eccentric connection to a pump motor;

FIG. 2 is a cross-sectional view of a multi-stroke accumulator linear piston/cylinder-style pump of the present disclosure, with an eccentric connection between the rigid piston and the motor shaft;

FIG. 3 is an exploded view of a rigid linear piston/cylinder and a multi-directional sealing gasket assembly of the pump of the present disclosure;

FIG. 4 is an exploded view of a rigid linear piston and eccentric roller bearing of the pump of the present disclosure, with a motor and a multi-directional sealing gasket;

FIG. 5 is an end view of a bearing insert of the linear piston of the pump of the present disclosure, with an offset-axis of the bearing insert in a first position, consistent with the piston's bottom-dead-center (or home) position;

FIG. 6 is an end view of the bearing insert of the linear piston of the pump of the present disclosure, with the offset-axis of the bearing insert in a second, intermediate position, consistent with the piston's eccentric (or tilted) position;

FIG. 7 is an end view of the bearing insert of the linear piston of the pump of the present disclosure, with the offset-axis of the bearing insert in a third position, consistent with the piston's top-dead-center (or chamber-evacuated) position;

FIG. 8 is a motor-end view of the bearing insert of the linear piston of the pump of the present disclosure, illustrating a shaft receptacle of the bearing insert in a first position, co-axial with the first position of the offset-axis of the bearing insert illustrated in FIG. 5, consistent with the piston's bottom-dead-center (or home) position;

FIG. 9 is a motor-end view of the bearing insert of the linear piston of the pump of the present disclosure, illustrating the shaft receptacle of the bearing insert in a second position, co-axial with the second position of the offset-axis of the bearing insert illustrated in FIG. 6, consistent with the piston's eccentric position;

FIG. 10 is a motor-end view of the bearing insert of the linear piston of the pump of the present disclosure, illustrating the shaft receptacle of the bearing insert in a third position, co-axial with the third position of the offset-axis of the bearing insert illustrated in FIG. 7, consistent with the piston's top-dead-center position;

FIG. 11 is an end view of a piston, bearing, bearing insert, and multi-directional gasket of the pump of the present disclosure, illustrating in solid lines the bearing insert in its first position and the piston in its corresponding bottom-dead-center (or home) position, and illustrating in phantom lines the second and third positions of the bearing insert (and respective corresponding eccentric and top-dead-center positions of the piston);

FIG. 12 is an end view of a piston, bearing, bearing insert, and multi-directional gasket similar to FIG. 11, but illustrating in solid lines the bearing insert in its second position and the piston in its corresponding eccentric position, and illustrating in phantom lines the first and third positions of the bearing insert (and respective corresponding bottom-dead-center (or home) and top-dead-center positions of the piston);

FIG. 13 is an end view of a piston, bearing, bearing insert, and multi-directional gasket similar to FIGS. 11 and 12, but illustrating in solid lines the bearing insert in its third position and the piston in its corresponding top-dead-center position, and illustrating in phantom lines the first and second positions of the bearing insert (and respective corresponding bottom-dead-center (or home) and eccentric positions of the piston);

FIG. 14 is an end view of a piston-cylinder assembly of the present disclosure, with the cylinder in phantom lines, the bearing insert in its second position, and the piston in its corresponding eccentric position;

FIG. 15 is a schematic representation of an exemplary cycle of the pump of the present disclosure;

FIG. 16 is a perspective view of a motor and piston-cylinder assembly of the pump of the present disclosure;

FIG. 17 is a cross-sectional view of the piston-cylinder assembly of the pump of the present disclosure, taken along lines 17-17 of FIG. 16, with the bearing insert in its second position and the piston in its corresponding eccentric position;

FIG. 18 is a perspective view of the pump of the present disclosure;

FIG. 19 is another perspective view of the pump of the present disclosure, with a housing of the motor removed;

FIG. 20 is an exploded view of the motor and piston-cylinder assembly of the pump of the present disclosure;

FIG. 21 is a perspective view of the pump of the present disclosure, including a motor housing;

FIG. 22 is a graph of vacuum pressure vs. time (in units of mm Hg/seconds), comparing vacuum output from a conventional single-stroke pump, a conventional diaphragm pump, and a multi-stroke accumulator pump of the present disclosure;

FIG. 23 is a graph of milk flow rate (in units of grams/second) versus time (in minutes) of a pumping session utilizing a conventional accumulator breastpump (designated “18202 QUEEN” in FIG. 23) having four displacement chambers in multiple states of evacuation; and

FIG. 24 is a graph of milk flow rate (in units of grams/second) versus time (in minutes) of a pumping session utilizing a breastpump having a multi-stroke accumulator linear piston/cylinder-style pump of the present disclosure.

### DETAILED DESCRIPTION OF THE PREFERRED EMBODIMENTS

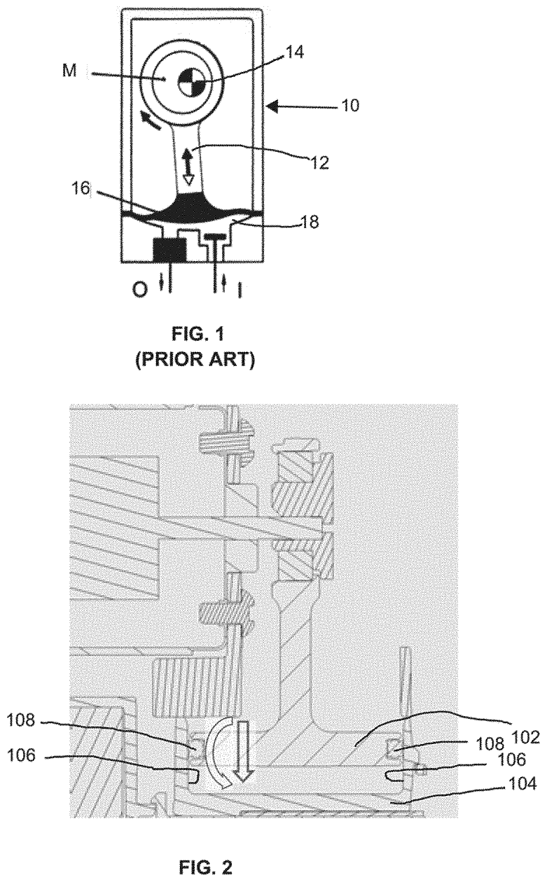

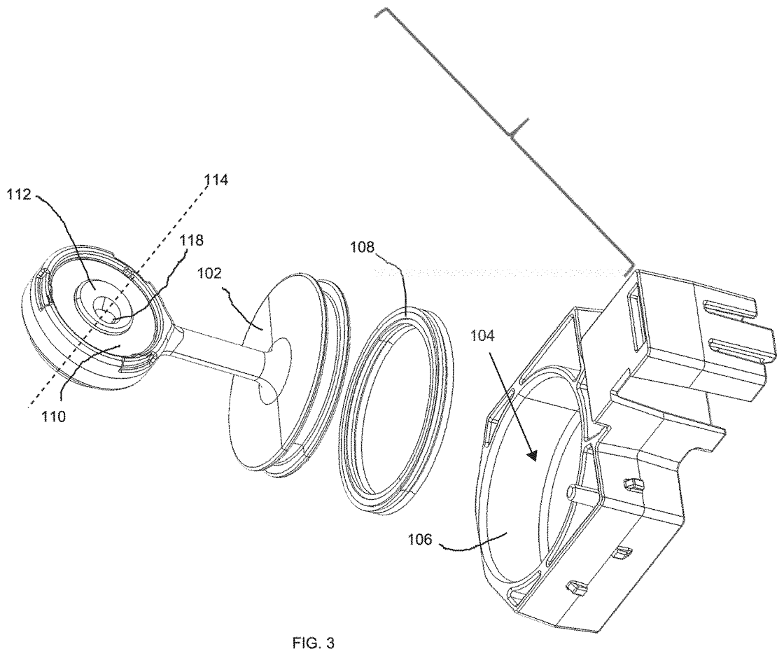

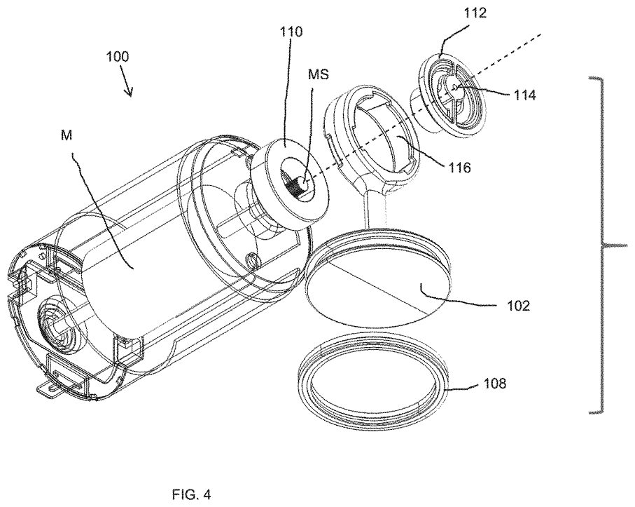

With reference to FIG. 1, a conventional diaphragm-style accumulator pump 10 is illustrated. A piston 12 of the diaphragm-style accumulator pump 10 has an eccentric engagement with an actuator motor (schematically represented in FIG. 1 by reference letter M) along a shaft 14. Through each revolution of the shaft 14, the piston 12 imparts first an up stroke, and next, a downstroke, to a diaphragm membrane 16. While this cyclical motion has the desired effect of building up pressure in a vacuum chamber 18, with each stroke, there are pressure losses across the membrane 16.

An accumulator-style pump 100 of the present disclosure is illustrated in cross-section in FIG. 2, and features a solid piston head 102 that is seated within a cylinder 104. The solid piston head 102 is actuated in an eccentric manner in order to deliver vacuum pulses to a surface to which vacuum pressure from the pulse is applied, such as a breast or a teat. The vacuum pressure may be applied directly, or to an intermediate surface such as a media separation membrane (not shown), used to maintain the integrity and sterility of the pump components and avoid contamination of collected fluid, such as breastmilk.

The solid piston head 102 engages a sidewall 106 of the cylinder 104. Because the solid piston head 102 is actuated in an eccentric manner, the piston head 102 does not maintain a perpendicular relationship with respect to the side wall 106 throughout its travel along the interior of the cylinder 104. Rather, the disc-like piston head 102 rocks from in a tilted manner as it moves up and down (as indicated by the curved and straight arrows in FIG. 2). In order to maintain sealed engagement of the piston head 102 with the sidewall 106 of the cylinder 104 throughout eccentric actuation of the piston head 102, it is preferred to use a multi-directional, low friction compression sealing gasket 108 having a non-circular cross-section configuration, such as the MULTISEAL® bi-directional lip style seal available from Precision Associates, Inc. of Minneapolis, Minn., USA.

In a particular embodiment, the eccentric actuation of the solid piston head 102 can be achieved utilizing a rotatable bearing 110 that receives a bearing insert 112 having an offset axis of rotation 114. The rotatable bearing 110 and bearing insert 112 are received in a complementary circular opening 116 of the piston head 102. A motor M of the pump 100 includes a motor shaft MS that is securely received in a shaft receptacle 118 of the bearing insert 112, which shaft receptacle 118 is coaxial with the offset axis of rotation 114 of the bearing insert 112.

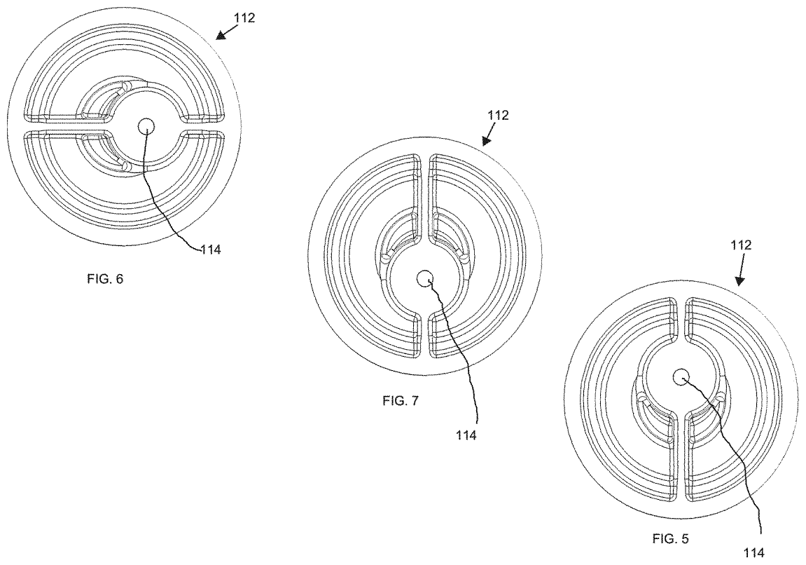

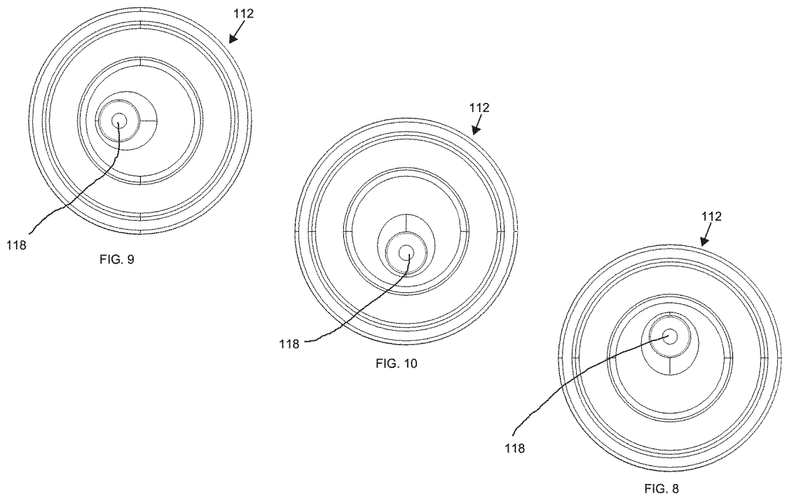

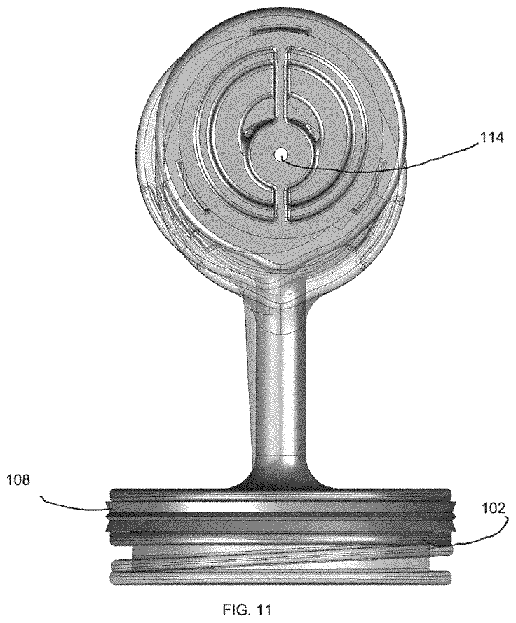

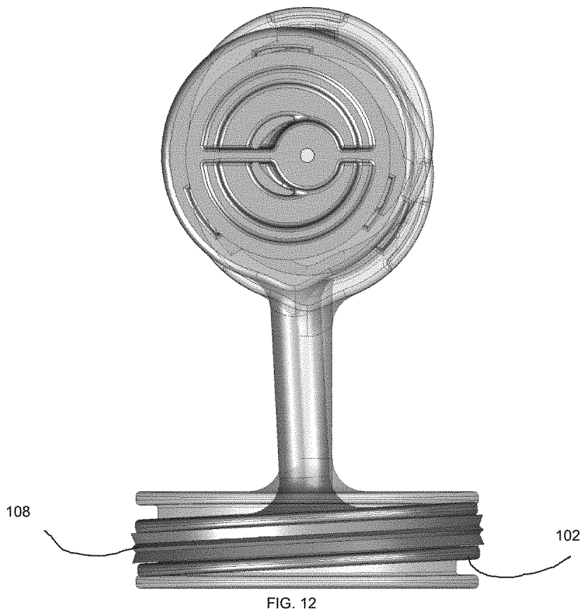

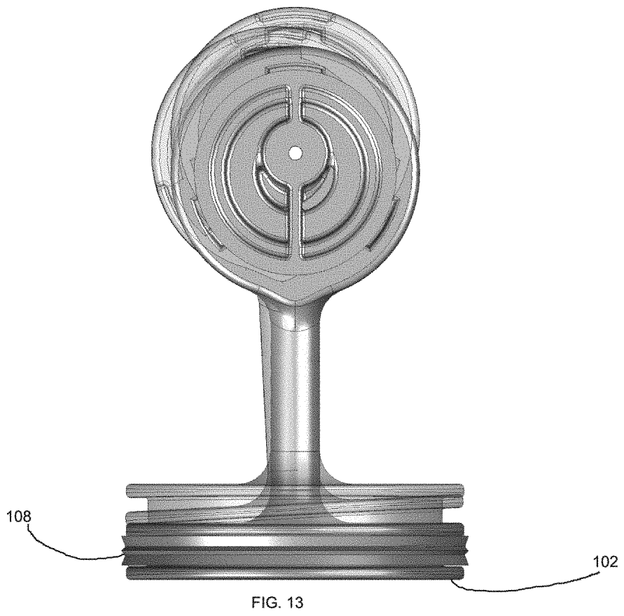

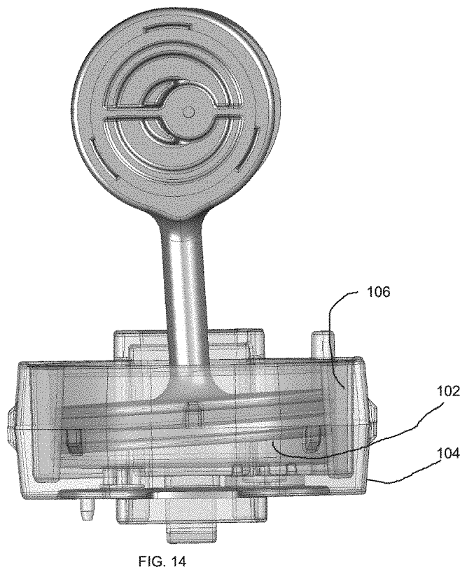

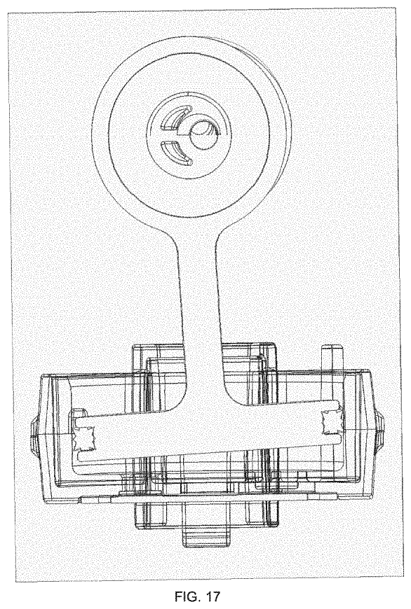

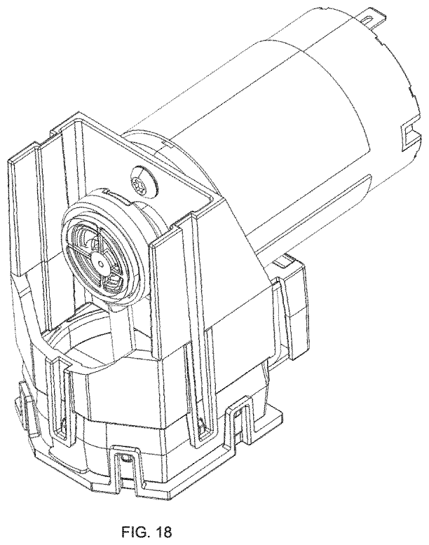

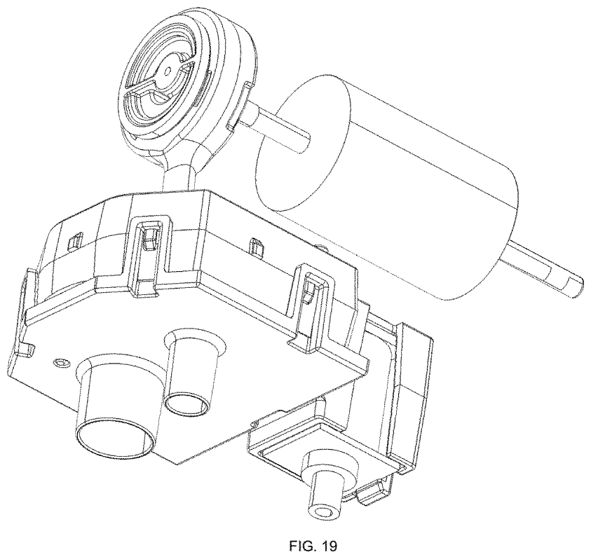

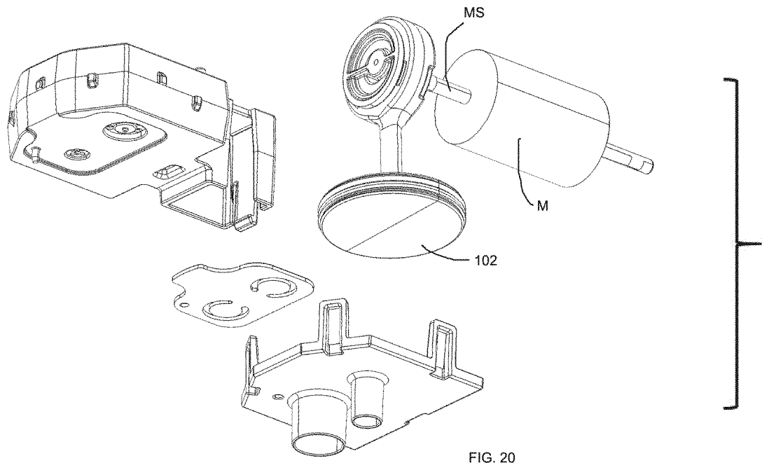

The sequence of actuation of the multi-stroke accumulator linear piston/cylinder-style pump of the present disclosure can be understood with reference to FIGS. 5-17. Three distinct positions of the bearing insert 112 are illustrated, respectively, in FIGS. 5, 6, and 7 (end views) and FIGS. 8, 9, and 10 (motor-end views). Corresponding views of the piston head 102 and sealing gasket 108 are illustrated in FIGS. 11, 12, and 13. FIGS. 5, 8, and 11 illustrate the bearing insert 112 in a first position, corresponding to a home or bottom-dead-center position of the piston head 102. FIGS. 6, 9, and 12 illustrate the bearing insert 112 in a second position, corresponding to an eccentric position of the piston head 102. FIGS. 7, 10, and 13 illustrate the bearing insert 112 in a third position, corresponding to a top-dead-center position of the piston head 102. While the piston head 102 and sealing gasket 108 are illustrated in solid lines in each of the various first, second, and third positions in FIGS. 11, 12, and 13, respectively, the alternate positions of the piston head 102 are illustrated in phantom lines in each of those figures.

With reference to FIGS. 12, 14, and 17, it can be appreciated that when the piston head 102 is in the eccentric position, the piston head 102 has moved not only vertically, but also to an angle relative to the sidewall 106 of the cylinder 104. As compared to conventional single-stroke pistons, the pump 100 of the present disclosure advantageously applies a vacuum pulse frequency over the cyclical vacuum frequency that would otherwise be applied had the piston head 102 only moved up and down once. The vacuum pulse can be attributed to the multiple rotations (approximately 20) of the piston head 102 relative to the sidewall 106 of the cylinder 104 during the cyclical vacuum cycle. Two check valves allow the incremental building of vacuum with each rotation until the target vacuum is achieved. The volume of air displaced during a single rotation of the piston is approximately 1/20^{th} of the volume of air displaced for a conventional single-stroke piston.

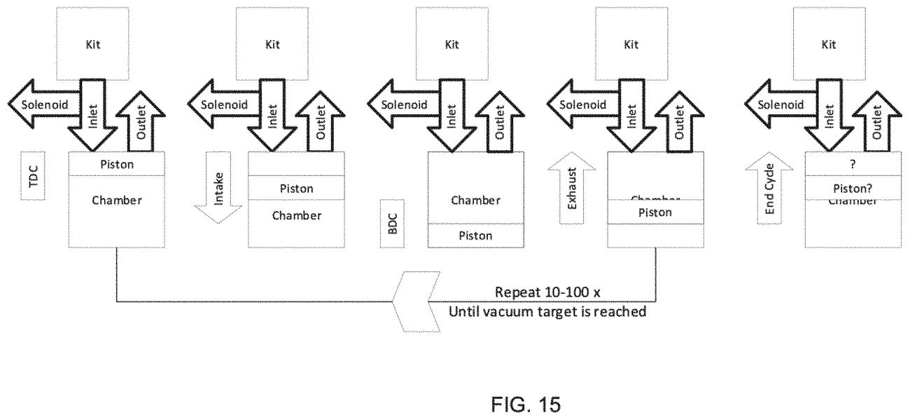

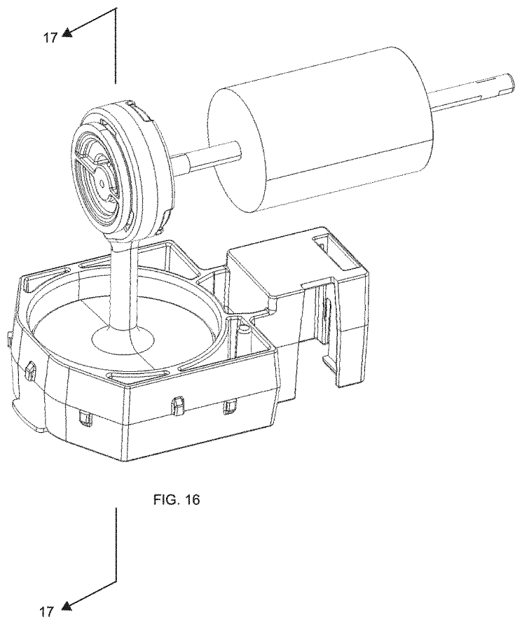

An exemplary cycle of the pump of the present disclosure is illustrated in FIG. 15. The following table indicates the status of each component of the cycle at a given state of the cycle:

| Ref. | State | Piston | Inlet | Outlet | Solenoid |
|---|---|---|---|---|---|
| 1 | Top Dead | 0 | 1 | 0 | 0 |
| | Center (TDC) | | | | |
| 2 | Intake | 1 | 1 | 0 | 0 |
| 3 | Bottom Dead | 0 | 1 | 0 | 0 |
| | Center (BDC) | | | | |
| 4 | Exhaust | 1 | 0 | 1 | 0 |
| 5 | End Cycle | 1 | 0 | 1 | 1 |

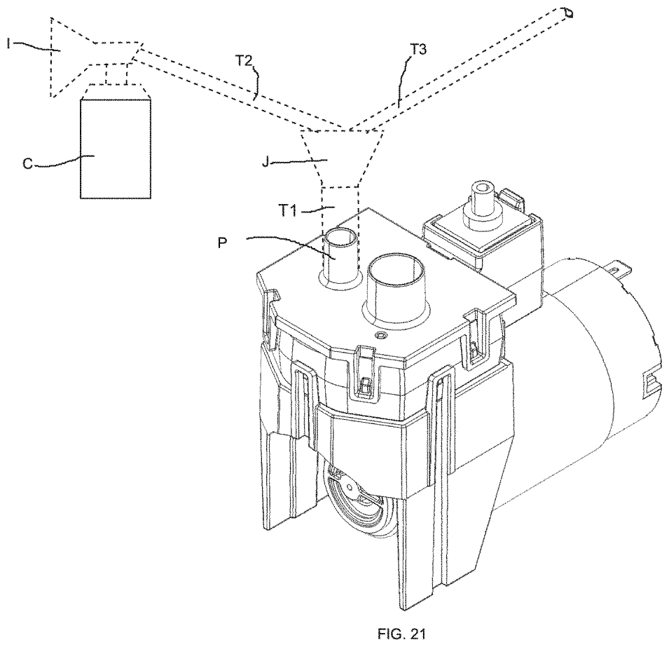

As illustrated in FIG. 21, the pump includes a port P that may be placed into fluid communication with a tubing system (illustrated in broken lines in the figure), including a first length of tubing T1, a junction J, a second length of tubing T2 that is in fluid communication with a first breastmilk collection kit K including a milk collection container C and an interface I, such as a breastshield, that receives the breast (not shown) and a media separation membrane (also not shown) to permit vacuum to be applied to the interior of the interface, and thus to the breast received therein, and permit collection of breastmilk, while isolating the tubing circuit from the breastmilk, thereby preventing contamination of the pump. The system can optionally further include, in order to provide simultaneous pumping, a third length of tubing T3 that is in fluid communication with a second breastmilk collection kit (not shown).

Other embodiments that are within the scope of the present disclosure include alternate structural configurations for a vacuum pump mechanism that achieve controllable vibration pulses. For instance, rather than having a piston that undergoes an eccentric movement, a vacuum pump having a piston that maintains a vertical orientation throughout its travel along the sidewall 106 of the cylinder 104, but employs a yoke, linkages, and/or direct action (such as a solenoid, a servo motor, or a DC stepper motor capable of pulsed actuation).

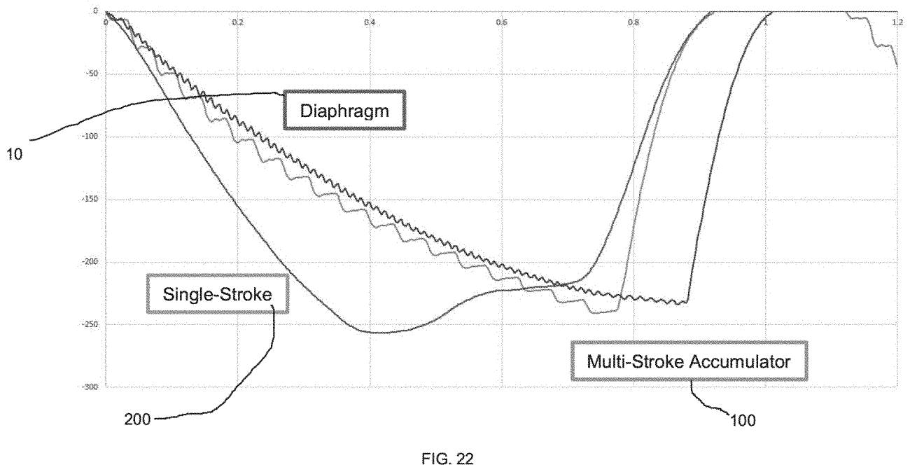

FIG. 22 graphically compares vacuum-versus-time plots of a conventional single-stroke pump 200 (which, as can be seen in the figure, generates a cyclical vacuum reflected by a smooth-lined vacuum curve), a conventional diaphragm-style accumulator pump 10, and a multi-stroke accumulator pump 100 of the present disclosure. While a diaphragm-style accumulator pump 10 can apply a non-smooth vacuum curve, the amplitude of the variations in vacuum pressure are de minimis, likely due to the dampening effect of the diaphragm membrane 16. Moreover, due to the deterioration of the diaphragm membrane 16 over time, such as due to fatigue, cracking, or tearing of the membrane 16, vacuum curves of a diaphragm-style accumulator pump 10 tend to smooth out over time. There is also a lack of precision and fundamental difficulty in the ability to repeatedly obtain predictable vacuum pressure variation with a diaphragm-style accumulator pump 10. The multi-stroke accumulator pump 100, by virtue of the eccentric movement of its rigid piston head 102 and lack of a diaphragm membrane 16, has the ability to be reliably tuned to a predictable vacuum frequency.

The motor M of the multi-stroke accumulator pump 100 of the present disclosure has an increased torque relative to motors employed with a conventional single-stroke pump 200 or a diaphragm-style accumulator pump 10, which is necessary to achieve the discernible, predictable vacuum pulses of sufficient amplitude to achieve advantageous results, as described in more detail below. A benefit of the increased torque of the motor M is that each vacuum pulse creates a distinct load on the motor shaft MS that is directly proportional to the current draw of the motor M. As such, the vacuum load can be accurately predicted based on the current draw.

Another benefit of the correlation between the current draw of the motor M and the resulting vacuum of the multi-stroke accumulator pump 100 of the present disclosure is that the pump 100 can be controlled by monitoring the current output of the motor M without the need to include a pressure monitor. To avoid applying too strong of a vacuum, a predetermined not-to-exceed current output of the motor M can be set.

A practical effect of the predictable vacuum pulse generated by the multi-stroke accumulator pump 100 of the present disclosure when employed in a breast pump is increased oxygenation of breast alveoli and improved milk removal, as compared to vacuum supplied by conventional single-stroke pumps 200 or diaphragm-style accumulator pumps 10. The combined cyclical vacuum frequency and vacuum pulse frequency applied by the multi-stroke accumulator pump 100 imparts a high-frequency oscillatory ventilation to the breast alveoli. In a manner similar to the treatment of respiratory distress syndrome through the application of HVOC, which utilizes vacuum pulse generation to provide increased oxygenation to lung alveoli, increased stimulation of the breast alveoli during the course of a breast pumping session is found to result in an increased volume of collected breastmilk.

Percent of available milk removed, or PAMR, is a metric used to quantify the volume of collected breastmilk.

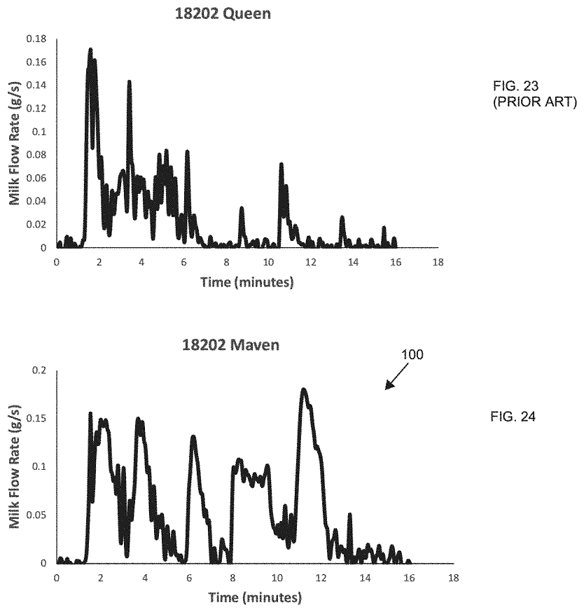

A comparison of FIGS. 23 and 24 graphically illustrates a measurable increase in milk flow rate during the course of a pumping session using a multi-stroke accumulator pump 100 of the present disclosure (FIG. 24), as compared to milk flow rate during the course of a pumping session by the same nursing mother using a conventional accumulator breastpump (FIG. 23).

Moreover, it is understood that breastmilk having higher fat concentration leads to more complete removal of the milk to a feeding infant, and a more effective pump. During the course of a pumping session, it is found that the viscosity of breastmilk increases toward the end of the pumping session, indicative of an increase in fat content of the breastmilk. However, with conventional cyclical vacuum breast pumps, the less-ventilated breast alveoli tend to close up, or the viscosity of the breastmilk otherwise impedes the secretion of further breastmilk through the breast alveoli, despite the epithelial milk-secreting cells having generated additional desirable high-nutrient, relatively high-fat breastmilk. This precious additional breastmilk, when not released through the breast alveoli, goes unused. Over time, the inability to remove the all available milk from the breast alveoli, whether through baby feeding, pumping, or some combination of the two, will diminish available milk supply. Thus, an advantage of the multi-stroke accumulator pump 100 of the present disclosure, and other vacuum pump mechanisms that achieve controllable vibration pulses which are within the scope of the present disclosure, is the provision of a device that facilitates complete or near-complete emptying of the breast alveoli, which promotes increased, or at least sustained, milk supply over time. Through the use of the multi-stroke accumulator pump 100 of the present disclosure, it is found that not only is increased breastmilk collection during a given pumping cycle achieved, but also, the collected breastmilk includes the higher fat content breastmilk that heretofore went uncollected by electric and manual breastpumps.

Yet a further benefit of the multi-stroke accumulator pump 100 of the present disclosure, and other vacuum pump mechanisms that achieve controllable vibration pulses which are within the scope of the present disclosure, is that the increase in milk flow rate during the course of a pumping session is achieved via application of less vacuum pressure. Practical benefits of being able to achieve increased milk collection with lower vacuum pressure include lower power requirements (facilitating use of less-costly power supplies, lighter power supplies, and/or rechargeable power supplies that require less time to re-charge or that maintain their charge longer before they require a recharging), and greater comfort for the nursing mother. The increased pumping efficiency also affords several benefits, such as faster pumping (and therefore shorter pumping sessions), greater breastmilk collection in a single pumping session, and, as discussed above, increased, or at least less-diminished, milk supply over time.

While the present disclosure is presented with human breastpumping as its primary use, milking machines for use in the dairy industry may be constructed with pumping mechanisms that utilize a multi-stroke accumulator pump made according to the teachings of this disclosure, and other vacuum pump mechanisms that achieve controllable vibration pulses, to achieve many, if not all, of the benefits described herein. For milking machines in the dairy industry, for instance, the interface I may instead be one that receives one or more bovine teats. A single pump of the present disclosure could be used in such settings in simultaneous pneumatic communication with more than two teats, and even with more than a single lactating cow, so as to milk a plurality of cows simultaneously.

A multi-stroke accumulator pump 100 may be operated at 0.1 mmHg or higher, and a frequency below 80 Hz. The frequency can be below 30 Hz, or in the range of 10-30 Hz.

While certain embodiments are described herein, variations may be made that are still within the scope of the appended claims.

What is claimed is:

1. A method for pumping milk comprising:
providing an interface within which a breast or teat is received to express milk;
providing a source of vacuum in communication with the interface; and
operating the source of vacuum to apply both a cyclical vacuum and a vacuum pulse frequency, generating a combined vacuum.

2. The method of claim 1, wherein in operating the source of vacuum, the combined vacuum has a frequency below 30 Hz.

3. The method of claim 1, wherein in operating the source of vacuum, the combined vacuum has a frequency in a range of 10-30 Hz.

4. An improved method of pumping milk, including applying a cyclical vacuum to a breast of a lactating woman, the improvement comprising:
additionally applying a vacuum pulse frequency over the cyclical vacuum.

5. The improved method of pumping milk of claim 4, wherein in additionally applying the vacuum pulse frequency over the cyclical vacuum, generating a combined vacuum having a net effective vacuum of at least 0.1 mmHg, and a frequency below 80 Hz.

6. The improved method of pumping milk of claim 5, wherein in additionally applying the vacuum pulse frequency over the cyclical vacuum, operating an eccentric piston pump.

7. A method of breastpumping comprising providing a first operational mode in which a cyclical vacuum is applied to a breast at a first vacuum pressure; and providing a second operational mode in which a vacuum pulse is selectively applied in addition to the cyclical vacuum, wherein a net negative pressure resulting from the combined cyclical vacuum and the vacuum pulse is effectively a second vacuum pressure that applies a lower suction force to the breast than the first vacuum pressure.

8. A method of enhancing expression of milk from breast alveoli, comprising:
providing a breastshield having a portion within which a woman's breast is received to express milk;
providing a source of vacuum in communication with the breastshield;
providing high frequency oscillatory ventilation to the breast alveoli by operating the source of vacuum to apply both a cyclical vacuum and a vacuum pulse frequency.

9. A method of pumping, comprising:
providing a pump that applies a vacuum pulse in addition to a cyclical vacuum, which combine to deliver a vacuum that varies in amplitude.

10. A breastpump comprising:
a vacuum source that applies both a vacuum pulse frequency and a cyclical vacuum.

11. The breastpump of claim 10, further comprising:
a drive motor having a motor shaft;
a cylinder;
a rigid piston disposed within the cylinder, the rigid piston eccentrically mounted on the motor shaft and being actuatable via rotation of the motor shaft to translate both vertically along an axis of the cylinder and at an angle;
one or more valves provided on a wall of the cylinder, the one or more valves permitting application of negative pressure that accumulates in the cylinder after a plurality of cycles of the rigid piston in the cylinder.

12. The breastpump of claim 11, wherein each vacuum pulse creates a distinct load on the motor shaft that is directly proportional to a current draw of the motor.

13. The breastpump of claim 12, further comprising a sensor that senses the effect on the current draw of the motor by the load applied to the motor shaft by each vacuum pulse, and a controller, the controller including a memory stored on a non-transitory computer readable medium that includes a target vacuum pressure, and a comparator that compares the sensed effect on the current draw of the motor from the sensor to the stored target vacuum pressure, and selectively opens or closes the one or more valves based on whether the comparator determines the effect on the current draw of the motor is above, below, or equal to, the target vacuum pressure.

14. The breastpump of claim 11, in further combination with at least one breastshield and tubing in pneumatic communication with the breastshield and one or more valves of the cylinder.

15. The breastpump of claim 11, in combination with one or more additional like breastpumps.
